(19) United States
(12) Patent Application Publication
(10) Pub. No.: US 2026/0240663 A1
(43) Pub. Date: Aug. 20, 2026

(54) IMPROVEMENTS IN AND RELATING TO SURGICAL INSTRUMENTS

(71) Applicant: DEPUY IRELAND UNLIMITED COMPANY, RINGASKIDDY (IE)

(72) Inventor: JAMES ANDERSON, YORK, NORTH YORKSHIRE (GB)

(21) Appl. No.: 19/113,301

(22) PCT Filed: Oct. 24, 2023
(86) PCT No.: PCT/EP2023/079592

§ 371 Date: Mar. 19, 2025

(30) Foreign Application Priority Data
Oct. 24, 2022 (GB) 2215748.1

(51) Int. Cl. A61F 2/46

(52) U.S. Cl. CPC A61F 2/4637; A61F 2/4603; A61F 2002/4627

(57) ABSTRACT

Orthopaedic load generating instruments, such as head to liner presses, kits including such instruments and methods of using such instruments are provided. The surgical instrument comprising: a handle element; a driven element mounting section connected to the handle element; one or more driven elements, the one or more driven elements including a driven element providing a proximal driver attachment location, the one or more driven elements including a driven element providing a proximal implant component contactor; and an instrument support section, the instrument support section including a first abutment surface and at least a second abutment surface; wherein the instrument has a driven axis, the driven axis corresponding to a driven element axis extending distally to proximally through one or more of the driven elements; wherein a first intersection defined between the driven axis and the first abutment surface forms a first angle and a second intersection defined between the driven axis and the second abutment surface forms a second angle, the second angle being different to the first angle. In this way, the instrument has a first state in which the driven axis has a first angular orientation relative to the vertical and the instrument has a second state in which the driven axis has a second, different, angular orientation relative to the vertical.

### TECHNICAL FIELD

The present disclosure relates generally to surgical instruments, particularly orthopaedic surgical instruments, kits including such instruments and methods of using such instruments. More particularly the present disclosure relates to orthopaedic load generating instruments, such as head to liner presses, kits including such instrument and methods of using such instrument. The disclosure also has wider relevance in other surgical instruments where loads need to be generated under user control of the orientation of the instrument.

### BACKGROUND

Joint arthroplasty is a well-known surgical procedure by which a diseased and/or damaged natural joint is replaced by a prosthetic joint. Examples include knee prosthesis and hip implants. During assembly of the prosthesis, for example a femoral implant and acetabulum implant, there is a need to apply loaf to components in the prosthesis to reduce them together and into their use position. Careful alignment and control of the load is desirable in this operation.

### SUMMARY

According to a first aspect of the disclosure there is provided a surgical instrument, the surgical instrument comprising:
a handle element;
a driven element mounting section connected to the handle element;
one or more driven elements, the one or more driven elements including a driven element providing a proximal driver attachment location, the one or more driven elements including a driven element providing a proximal implant component contactor; and
an instrument support section, the instrument support section including a first abutment surface and at least a second abutment surface;
wherein the instrument has a driven axis, the driven axis corresponding to a driven element axis extending distally to proximally through one or more of the driven elements;
wherein a first intersection defined between the driven axis and the first abutment surface forms a first angle and a second intersection defined between the driven axis and the second abutment surface forms a second angle, the second angle being different to the first angle.

The first intersection and/or second intersection and/or any and all further intersections can be defined by a projection of the respective abutment surface to the driven axis.

The first abutment surface and/or second abutment surface and/or any and all further abutment surfaces may be instrument support location abutment surfaces, such as instrument support table surfaces.

The instrument may have a first state in which the first abutment surface is adapted to contact an instrument support location, such as an instrument support table surface. The instrument may have a second state in which the second abutment surface is adapted to contact an instrument support location, such as an instrument support table surface. The instrument may have a third state in which a third abutment surface is adapted to contact an instrument support location, such as an instrument support table surface. The instrument may have one or more further states in which one of one or more further abutment surfaces are adapted to contact an instrument support location, such as an instrument support table surface.

The instrument may have a first state in which the driven axis has a first angular orientation relative to the vertical. The instrument may have a second state in which the driven axis has a second, different, angular orientation relative to the vertical. The instrument may have a third state in which the driven axis has a third, different, angular orientation relative to the vertical. The instrument may have one or more further states in which the driven axis has a further, different, angular orientation relative to the vertical.

The instrument may have a first state in which the driven axis is vertical +/−5°. The instrument may have a second state in which the driven axis is at 30° to the vertical +/−5°. The instrument may have a third state in which the driven axis is at 45° to the vertical +/−5°. The instrument may have one or more further states in which the driven axis is off set from the vertical by more than 55° to the vertical +/−5°. The instrument may have one or more further states in which the driven axis is off set from the vertical in the opposite angular direction to the first state and/or second state and/or third state.

The instrument may have a first state in which the second abutment surface and/or a third abutment surface and/or any further abutment surfaces are not adapted to contact an instrument support location, such as an instrument support table surface. The instrument may have a second state in which the first abutment surface and/or a third abutment surface and/or any further abutment surfaces are not adapted to contact an instrument support location, such as an instrument support table surface. The instrument may have a third state in which the first abutment surface and/or a second abutment surface and/or any further abutment surfaces are not adapted to contact an instrument support location, such as an instrument support table surface. The instrument may have a further state in which the first abutment surface and/or second abutment surface and/or a third abutment surface are not adapted to contact an instrument support location, such as an instrument support table surface.

The handle element may be adapted for a single hand of the user. The handle element may be adapted for right-handed and/or left-handed use by the user.

The handle element may be substantially cylindrical. The handle element may have a tapering cross-section, for instance tapering to a smaller cross-section in a superior direction.

The handle element may be integrally provided with the driven element mounting section and/or integrally provided with the instrument support section.

The handle element may extend along a handle element axis. The handle element axis may have a constant angle of intersection with the driven axis. The handle element axis may have a constant angle of intersection with the driven axis in the first state and/or second state and/or third state and/or one or more further states. The handle element axis may have a constant angle of intersection with the instrument support section, for instance in the first state and/or second state and/or third state and/or one or more further states. The handle element axis may have a constant angle of intersection with the first abutment surface, for instance in the first state and/or second state and/or third state and/or one or more further states of intersection. The handle element axis may have a constant, different, angle of intersection with the second abutment surface, for instance in the first state and/or second state and/or third state and/or one or more further states of intersection. The handle element axis may have a constant, different, angle of intersection with the third abutment surface, for instance in the first state and/or second state and/or third state and/or one or more further states of intersection. The handle element axis may have a constant, different, angle of intersection with the one or more further abutment surfaces, for instance in the first state and/or second state and/or third state and/or one or more further states of intersection.

In a first state, the handle element axis may be at 45° to the vertical +/−5°. In a second state, the handle element axis may be at 30° to the vertical +/−5°. In a third state, the handle element axis may be vertical +/−5°.

The driven element mounting section may be substantially cylindrical. The driven element mounting section may have a substantially constant cross-section, for instance along at least 50% of the axial length of the driven element mounting location.

The driven element mounting section may be integrally provided with the handle element.

The driven element mounting section may extend along a driven element mounting section axis. The driven element mounting section axis may be coaxial with the instrument driven axis. The driven element mounting section axis may be coaxial with the instrument driven axis in the first state and/or second state and/or third state and/or one or more further states.

The driven element mounting section axis may have a constant angle of intersection with the instrument support section, for instance in the first state and/or second state and/or third state and/or one or more further states. The driven element mounting section axis may have a constant angle of intersection with the first abutment surface, for instance in the first state and/or second state and/or third state and/or one or more further states of intersection. The driven element mounting section axis may have a constant, different, angle of intersection with the second abutment surface, for instance in the first state and/or second state and/or third state and/or one or more further states of intersection. The driven element mounting section axis may have a constant, different, angle of intersection with the third abutment surface, for instance in the first state and/or second state and/or third state and/or one or more further states of intersection. The driven element mounting section axis may have a constant, different, angle of intersection with the one or more further abutment surfaces, for instance in the first state and/or second state and/or third state and/or one or more further states of intersection.

In a first state, the driven element mounting section axis may be vertical +/−5°. In a second state, the driven element mounting section axis may be at 30° to the vertical +/−5°. In a third state, the driven element mounting section axis may be at 45° to the vertical +/−5°.

A single driven element may be provided in which the driven element providing the proximal driver attachment location is the same as the driven element providing the distal implant component contactor.

The driven element providing the distal implant component connector may reciprocate relative to the driven element mounting section, particularly along the instrument driven axis. The driven element providing the distal implant component connector may be reciprocally mounted in the driven element mounting section.

The proximal driver attachment location may be provided on a different driven element to the driven element providing the distal implant component contactor.

The driven element providing the proximal driver attachment location may rotate relative to the driven element mounting section, particularly about the instrument driven axis. The driven element providing the proximal driver attachment location may be rotatably mounted in the driven element mounting section.

A drive conversion mechanism may be provided between the driven element providing the proximal driver attachment location and the driven element providing the proximal driver attachment location. The drive conversion mechanism may provide a rotational to reciprocating conversion. The drive conversion mechanism may be provided within the driven element mounting section.

The distal implant component contactor may be distal relative to the one or more driven elements. The distal implant component contactor may be proximal relative to a second implant component contactor associated with the instrument support section.

The distal implant component contactor may be adapted to receive a prosthesis component, such as a liner. The distal implant component contactor may be dished or recessed, particularly in the superiorly. The distal implant component contactor may provide an internal surface, for instance facing inferiorly, which is substantially complimentary to at least a part of the prosthesis component.

The distal driver attachment location may be adapted to receive or be received in a driver device.

The instrument support section may be adapted to support the instrument in cooperation with a non-instrument surface, such as a table surface or a prosthesis component surface.

The instrument support section may be substantially rectilinear in cross-section. The instrument support section may have a substantially constant cross-section, for instance over at least 50% of its length.

The instrument support section may be integrally provided with the handle element.

The instrument support section may include a first abutment surface and a second abutment surface and at least a third abutment surface.

Within the instrument support section, the first abutment surface may be more distal to the handle element than the second abutment surface and/or third abutment surface. Within the instrument support section, the second abutment surface may be more distal to the handle element than the third abutment surface and/or more proximal to the handle element than the first abutment surface. Within the instrument support section, the third abutment surface may be more proximal to the handle element than the first abutment surface and/or second abutment surface.

A transition surface may be provided between an abutment surface and another abutment surface. One or more or all of the transition surfaces may be curved. A transition surface may be provided between the first abutment surface and the second abutment surface. A transition surface may be provided between the second abutment surface and the third abutment surface. A transition surface may be provided between the third abutment surface, or a further abutment surface, and the handle element.

One or more or all of the abutment surfaces may be longer than they are wide. The length may be considered distally-proximally relative to the handle element. The width may be considered perpendicular to the length and perpendicular to the instrument driven axis. The length of two or more or all of the abutment surfaces may be the same +/−20%. The width of two or more or all of the abutment surfaces may be the same +/−20%. The depth of two or more or all of the abutment surfaces may be the same +/−20%. The depth may be perpendicular to the length and to the width.

The first abutment surface and/or second abutment surface and/or third abutment surface and/or further abutment surfaces may have similar surface profiles. A surface profile may be similar to another surface profile in having the same length +/−10% and/or the same width +/−10%. A surface profile may be similar to another surface profile in having the same surface shape over at least 80% of the surface.

The first abutment surface and/or second abutment surface and/or third abutment surface and/or further abutment surfaces may include a substantially planar surface section. The first abutment surface and/or second abutment surface and/or third abutment surface and/or further abutment surfaces may be a substantially planar surface section.

A surface may be substantially planar where all elements of the surface are within a deviation of less than 1 mm, for instance less than 0.5 mm, potentially less than 0.3 mm or possibly less than 0.2 mm, relative to a virtual perfectly planar surface. The virtual perfect planar surface may pass through one or more elements of the surface.

A surface may be a substantially planar surface where the separation between a virtual perfectly planar surface on which the most superior element of the surface lies and a second virtual perfectly planar surface on which the most inferior element of the surface lies is less than 1 mm separation, for instance less than 0.5 mm, potentially less than 0.3 mm or possibly less than 0.2 mm.

The first abutment surface and/or second abutment surface and/or third abutment surface and/or further abutment surfaces may provide a planar surface section or be a planar surface section, wherein the surface section is planar in a plane that is perpendicular to an instrument plane. The instrument plane may be the plane in which two or more or all of the handle element and/or the driven element mounting section and/or the one or more driven elements and/or the instrument support section lie or extend.

The first abutment surface, particularly a planar surface section thereof, may be inclined relative to the second abutment surface, particularly a planar surface section thereof, by an angle of 30°+/−10 °, for instance, +/−5°. The first abutment surface, particularly a planar surface section thereof, may be inclined relative to the third abutment surface, particularly a planar surface section thereof, by an angle of 45°+/−10 °, for instance, +/−5°. The second abutment surface, particularly a planar surface section thereof, may be inclined relative to the third abutment surface, particularly a planar surface section thereof, by an angle of 15°+/−10 °, for instance, +/−5°. The second abutment surface, particularly a planar surface section thereof, may be inclined relative to the first abutment surface, particularly a planar surface section thereof, by an angle of 30°+/−10 °, for instance, +/−5°.

A further abutment surface may be provided with an inclination, relative to the second abutment surface and/or relative to the third abutment surface, which is less than that of the first abutment surface. A further abutment surface may be provided with an inclination that is intermediate that of the second abutment surface and/or the third abutment surface relative to the first abutment surface. A further abutment surface may be provided with an inclination, relative to the first abutment surface and/or relative to the second abutment surface, which is greater than that of the third abutment surface.

The instrument may have a first abutment surface which is perpendicular to the driven axis +/−5°. The instrument may have a second abutment surface which is angled at 60°+/−5 ° to the driven axis. The instrument may have a third abutment surface which is angled at 45° +/−5° to the driven axis. The instrument may have one or more further abutment surfaces angled at less than 55°+/−5 ° to the driven axis.

The instrument may have a first state in which the first abutment surface is the abutment surface used to contact a non-instrument surface. The instrument may have a second state in which the second abutment surface is the abutment surface used to contact a non-instrument surface. The instrument may have a third state in which the third abutment surface is the abutment surface used to contact a non-instrument surface. The instrument may have one or more further states in which one for the further abutment surfaces is the abutment surface used to contact a non-instrument surface.

The instrument support section may be connected to a second implant component contactor. The second implant component contactor may be provided at the distal end, relative to the handle element, of the instrument support section.

The second implant component contactor may be adapted to receive a second prosthesis component, such as a head. The second implant component contactor may be shaped to engage with and/or support the second prosthesis component, for instance a cone or the like.

The second implant component contactor may contact or engage with the instrument support section, particularly at the distal end thereof relative to the handle element. The second implant component connector may pass between two parts of the instrument support section or an element provided thereon The two parts may be defined by a forked element of the instrument support section or of the element provided thereon.

The distal implant component contactor and second implant contactor may be coaxially provided. The first prosthetic component and the second prosthetic component may be coaxially provided when positioned on the distal implant component contactor and second implant contactor.

The separation of the distal implant component contactor and second implant contactor may be variable. The separation may be increased or decreased by a driver device.

Two or more or all of the different angular orientations for the instrument relative to the vertical may lie in the same plane. Two or more or all of the different angular orientations for the instrument relative to the vertical may lie in the same plane in the first state and/or second state and/or third state and/or one or more or all further states. The same plan may be the instrument plane.

Two or more or all of the handle element and/or the driven element mounting section and/or the one or more driven elements and/or the instrument support section may lie in the same plane. Two or more or all of the handle element and/or the driven element mounting section and/or the one or more driven elements and/or the instrument support section may lie in the same plane in the first state and/or second state and/or third state and/or one or more or all further states. The same plan may be the instrument plane.

The first aspect of the disclosure may include any of the features, options or possibilities set out elsewhere in this document, including in the other aspects of the disclosure.

According to a second aspect of the invention there is provided a kit, the kit comprising:
a surgical instrument;
a driver device;
wherein the surgical instrument comprises:
a handle element;
a driven element mounting section connected to the handle element;
one or more driven elements, the one or more driven elements including a driven element providing a proximal driver attachment location, the one or more driven elements including a driven element providing a proximal implant component contactor; and
an instrument support section, the instrument support section including a first abutment surface and at least a second abutment surface;
wherein the instrument has a driven axis, the driven axis corresponding to a driven element axis extending distally to proximally through one or more of the driven elements;
wherein a first intersection defined between the driven axis and the first abutment surface forms a first angle and a second intersection defined between the driven axis and the second abutment surface forms a second angle, the second angle being different to the first angle.

The driver device may be a rotary driver device, such as a power tool. The driver device may be a linear device, such as a reciprocal device. The driver device may be a ratchet. The driver device may include a ratchet mechanism and/or one or more arms to operate the driver device.

The driver device may include a driver handle section. The driver device may include a user interface to start or stop operation.

The driver device may have a driver device axis. The driver device axis may be coaxial with the axis of one or more or all of the driven elements. The drive device axis may be parallel to or coaxial with the instrument driven axis.

The driver handle section may define a user location, for instance the centre of the section held by the user.

The second aspect of the disclosure may include any of the features, options or possibilities set out elsewhere in this document, including in the other aspects of the disclosure.

According to a third aspect of the invention there is provided a method of assembly of a multi-component surgical implant, the method comprising:
providing at least two components of the multi-component surgical implant;
providing a surgical instrument comprising:
a handle element;
a driven element mounting section connected to the handle element;
one or more driven elements, the one or more driven elements including a driven element providing a proximal driver attachment location, the one or more driven elements including a driven element providing a proximal implant component contactor; and
an instrument support section, the instrument support section including a first abutment surface and at least a second abutment surface;
wherein the instrument has a driven axis, the driven axis corresponding to a driven element axis extending distally to proximally through one or more of the driven elements;
wherein a first intersection defined between the driven axis and the first abutment surface forms a first angle and a second intersection defined between the driven axis and the second abutment surface forms a second angle, the second angle being different to the first angle;
providing a first component of the surgical implant on the instrument support section; providing a second components of the surgical implant on the proximal implant component contactor;
abutting a chosen one of the abutment surfaces with a non-instrument surface;
driving a driven element to reduce the separation of the second component relative to the first component and push the second component and the first component into an assembled form.

The method may provide that the instrument support section supports the instrument in cooperation with the non-instrument surface. The non-instrument surface may be a table surface.

The method may provide that the first component and the second component are placed in cooperation with one another outside of a patient. The method may provide that the first component and the second component are placed in cooperation with one another with neither component in contact with a patient or implanted in a patient. The method may provide that the first component and the second component are placed in cooperation with one another outside of a patient in an assembly method.

The multi-component surgical implant may be an orthopaedic implant. The multi-component surgical implant may be a hip prosthesis. The multi-component surgical implant may be a shoulder prosthesis. The multi-component surgical implant may be a knee prosthesis. The first component may be a liner. The second component may be head.

The method may include moving the instrument to change the abutment surface abutting with the non-instrument surface.

The method may include connecting a driver device to the proximal driver attachment location. The method may include activating a driver device to drive a driven element. The driver device may include a driver handle section, potentially with the driver handle section defining a user location in the environment of the instrument. The method may include changing the abutment location used to change the user location in the environment of the instrument. The method may include changing the abutment location used to change the inclination of the driven axis.

The third aspect of the disclosure may include any of the features, options or possibilities set out elsewhere in this document, including in the other aspects of the disclosure.

According to a fourth aspect of the invention there is provided a method of surgery using a multi-component surgical implant, the method comprising:
providing at least two components of the multi-component surgical implant;
providing a surgical instrument comprising:
a handle element;
a driven element mounting section connected to the handle element;
one or more driven elements, the one or more driven elements including a driven element providing a proximal driver attachment location, the one or more driven elements including a driven element providing a proximal implant component contactor; and
an instrument support section, the instrument support section including a first abutment surface and at least a second abutment surface;
wherein the instrument has a driven axis, the driven axis corresponding to a driven element axis extending distally to proximally through one or more of the driven elements;
wherein a first intersection defined between the driven axis and the first abutment surface forms a first angle and a second intersection defined between the driven axis and the second abutment surface forms a second angle, the second angle being different to the first angle;
providing a first component of the surgical implant on the instrument support section providing a second components of the surgical implant on the proximal implant component contactor;
abutting a chosen one of the abutment surfaces with a non-instrument surface;
driving a driven element to reduce the separation of the second component relative to the first component and push the second component and the first component into an assembled form.

The method may provide that the instrument support section supports the instrument in cooperation with a non-instrument surface. The non-instrument surface may be a part of a prosthesis or implant in-situ in a patient.

The method may provide that the first prosthesis component and the second prosthesis component are placed in cooperation with one another inside of a patient. The method may provide that the first prosthesis component and the second prosthesis component are placed in cooperation with one another with one or both prosthetic components in contact with a patient or implanted in a patient. The method may provide that the first prosthesis component and the second prosthesis component are placed in cooperation with one another in a surgical method step.

The multi-component surgical implant may be an orthopaedic implant. The multi-component surgical implant may be a hip prosthesis. The multi-component surgical implant may be a shoulder prosthesis. The multi-component surgical implant may be a knee prosthesis. The first component may be a liner. The second component may be head.

The method may include moving the instrument to change the abutment surface abutting with the non-instrument surface.

The method may include connecting a driver device to the proximal driver attachment location. The method may include activating a driver device to drive a driven element. The driver device may include a driver handle section, potentially with the driver handle section defining a user location in the environment of the instrument. The method may include changing the abutment location used to change the user location in the environment of the instrument. The method may include changing the abutment location used to change the inclination of the driven axis.

The fourth aspect of the disclosure may include any of the features, options or possibilities set out elsewhere in this document, including in the other aspects of the disclosure.

### BRIEF DESCRIPTION OF THE DRAWINGS

Various embodiments of the disclosure will now be described, by way of example only, and with reference to the accompanying figures in which:

FIG. 1 is a prior art head to liner press instrument;

FIG. 2a is a head to liner press according to an embodiment of the disclosure in one state;

FIG. 2b is the head to liner press of FIG. 2a in a second state;

FIG. 2c is the head to liner press of FIG. 2c in a third state;

FIG. 3 is a further view of the head to liner press of FIG. 2a-2c; and

FIG. 4 is a schematic illustration of the interaction of a head to liner press with the immediate environment and the use location influencing factors.

### DETAILED DESCRIPTION OF THE DRAWINGS

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

Terms representing anatomical references, such as anterior, posterior, medial, lateral, superior, inferior, et cetera, may be used throughout the specification in reference to the orthopaedic implants or prostheses and surgical instruments described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise.

During hip replacement surgery, a new femoral head is often implanted. As part of that procedure, a liner assembly needs to be reduced onto the head. Control of the force applied and the orientation of the force applied are important to give proper head and liner assembly.

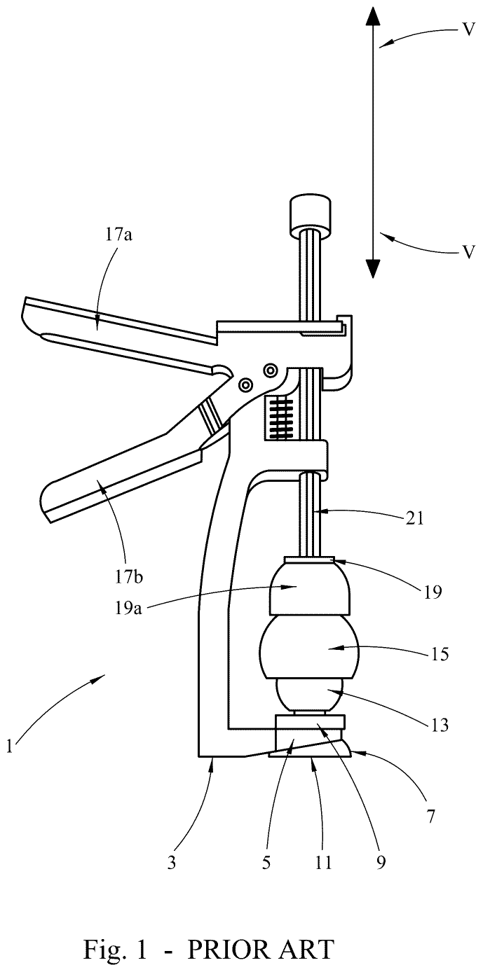

A head to liner press 1 of a prior art instrument type is shown in FIG. 1. The head to liner press 1 can be used for table top assembly or in-situ assembly of the liner and head combination. In FIG. 1, assembly on table is being provided. The head to liner press 1 is held vertically, direction V-V, on the table with a foot 3 of the press 1 resting on the table. The foot 3 has a forked section 5 at the end 7. A support cone 9 and a clamping ring 11 are screwed together, with screw threaded projection on the clamping ring 11 extending up between the arms of the forked section 5.

A head 13 can then be positioned on the support cone 9 and a liner 15 can be positioned on the head 13. By squeezing the handles 17a, 17b of the press 1 together, releasing and repeating, the end plate 19 and cup 19a on the end of the shaft 21 can be reduced onto the liner 15 and further movement reduces the liner 15 onto the head 13. When correctly assembled the liner 15 will rotate freely on the head 13.

The type of head to liner press 1 described above can also be used to form the assembly in-situ with the forked section 5 around the stem neck and under the head of the implant. The liner 15 is then positioned in opposition to head and the two are reduced together.

In either assembly modes, the press 1 has to apply the necessary force whilst the head 13 and the liner 15 are carefully aligned prior to and during loading. Hence, the stability of the press 1 and its ease of holding by the user in the required position are important considerations. At the same time, the press 1 needs to deliver loads of 800N to 1500N in normal use and so material effort by the user is required.

It is desirable to make use more comfortable for the user and to allow correct alignment and force application without undue effort or strain. Ergonomic improvements in the design and operation are possible and desirable.

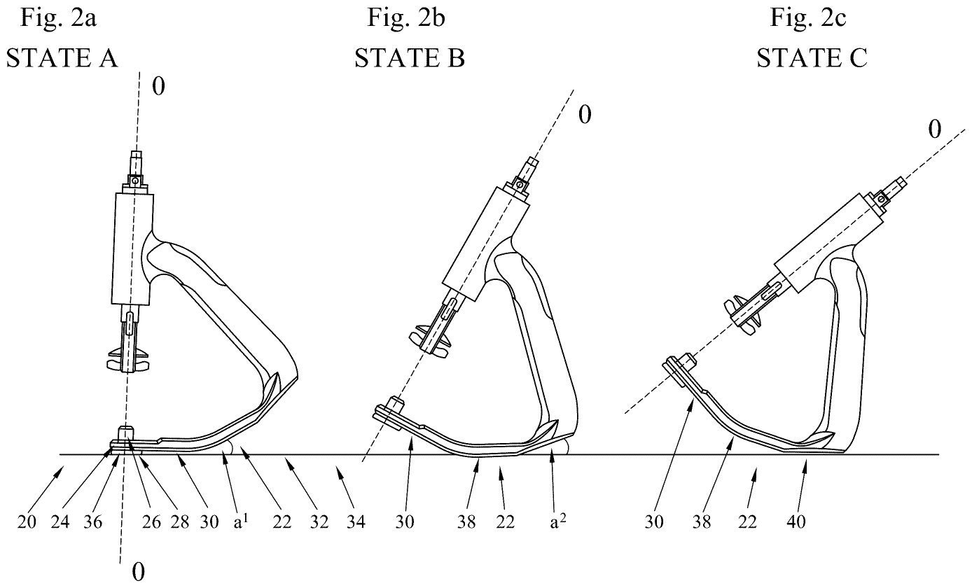

An instrument 20 according to one embodiment of the disclosure is shown in FIG. 2a in a first orientation, and hence first state A.

The instrument 20 has a foot section 22 which ends in a forked section 24. The forked section 24 allows a projection 26 from a clamping ring 28 to extend up between the arms of the forked section 24.

The foot section 22 has a first planar base section 30 which provides a first abutment surface to engage with the upper surface 32 of a table 34 in use. As shown, the first planar base section 30 is co-planar with the base part 36 of the forked section 24. The base part 36 could be recessed proximally relative to the first planar base section 30 so as to accommodate the clamping ring 28 above the plane of the upper surface 32 of the table 34.

With the first planar base section 30 on the upper surface 32 of the table 34, the operative axis O-O of the instrument 20 is vertically aligned. The planar nature of the base section 30 assists in maintaining the orientation of the instrument 20 and hence of the force applied during use. The width of the first planar base section 30 resists movement in one plane and the length resists movement in a perpendicular plane. The instrument 20 does not need to be freestanding in any of the states, but stable.

The foot section 22 also includes a second planar base section 38 towards the middle of the foot section 22. The second planar base section 38 has an angle a¹ of 30° relative to the first planar base section 30. As shown in FIG. 2b, the instrument 20 is in a second orientation and second state B with the second planar base section 38 resting on the upper surface 32 of the table 34. This presents the operative axis O-O at an angle of 30° relative to the vertical.

As a consequence of the different orientation in the second state B, the instrument 20 is easier to use for shorter users or when higher tables 34 are involved. The drive mechanism, as detailed further below, is brought closer to the edge of the table 34 and less inward reach is required and less height in the reach is required to use the instrument 20.

The foot section 22 also includes a third planar base section 40 towards the end of the foot section 22 away from the forked section 24. The third planar base section 40 has an angle a² of 15° relative to the second planar base section 38. As shown in FIG. 2 c, the instrument 20 is in a third orientation and third state C with the third planar base section 40 resting on the upper surface 32 of the table 34. This presents the operative axis O-O at an angle of 45° relative to the vertical.

As a consequence of the further different orientation in the third state C, the instrument 20 is even easier to use for shorter users or when higher tables 34 are involved. The drive mechanism, as detailed further below, is brought out over the edge of the table 34 and still less inward reach is required and still less height in the reach is required to use the instrument 20.

As a result of the three stable states, A, B and C, provided, the user has the choice of three different orientations for the instrument. Each of the states and its different orientation also brings with it a different position and orientation for the drive mechanism employed. Overall, the approach enables the user to make use of the instrument with their wrist and/or shoulder not in a stress position.

The three orientations provided for offer sufficient range of orientations for a wide enough range of users and factors impacting upon use. Further orientations and states could be introduced by providing further planar surfaces or facets, to the three provided above, at further different angles. The use of planar surfaces, such as flat facets, is beneficial in optimising stability in each state.

In manner in which the shaft 21 and end plate 19 are advanced into contact with the liner 15 and then to reduce the liner 15 on to the head 13 is open to many possibilities. A ratchet style mechanism of the type used in the FIG. 1 approach is one such drive mechanism. This provides a drive mechanism which is orthogonally aligned relative to the driven shaft 21. Rotation of the shaft 21 relative to a fixed body is another option for providing the drive mechanism. This could use proximal handles on the shaft 21 which extend radially outward.

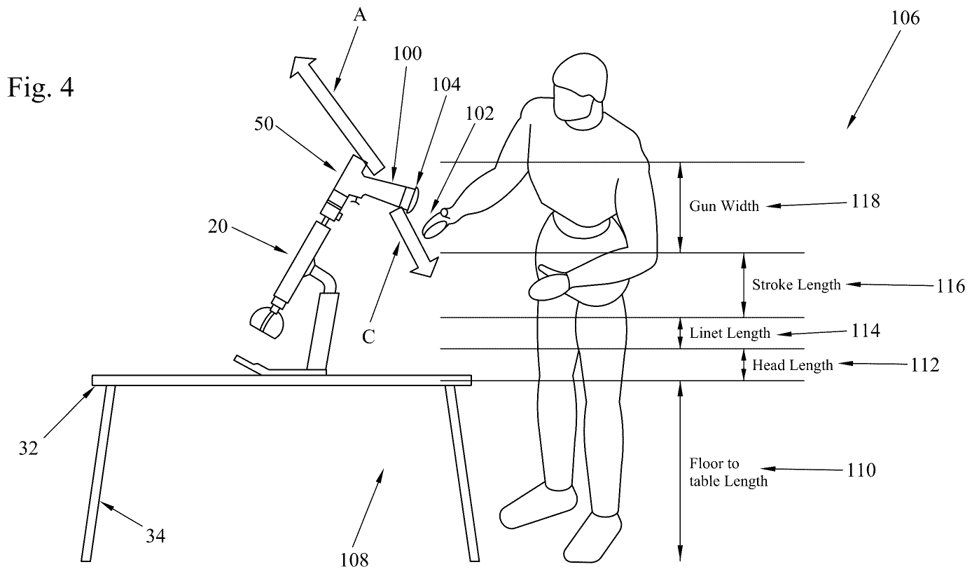

The drive mechanism illustrated in FIG. 4 is a powered screw drive 50. This is mounted on the proximal end 52 of the driven shaft 54 and so extends from the instrument 20 axially. This means that a longer construct arises and that gives greater adjustability to the orientation of the instrument. Furthermore, the handle 56 for such a drive mechanism is in an optimal position for holding and the drive mechanism can be fully activated and controlled without requiring material movement of the user's hand.

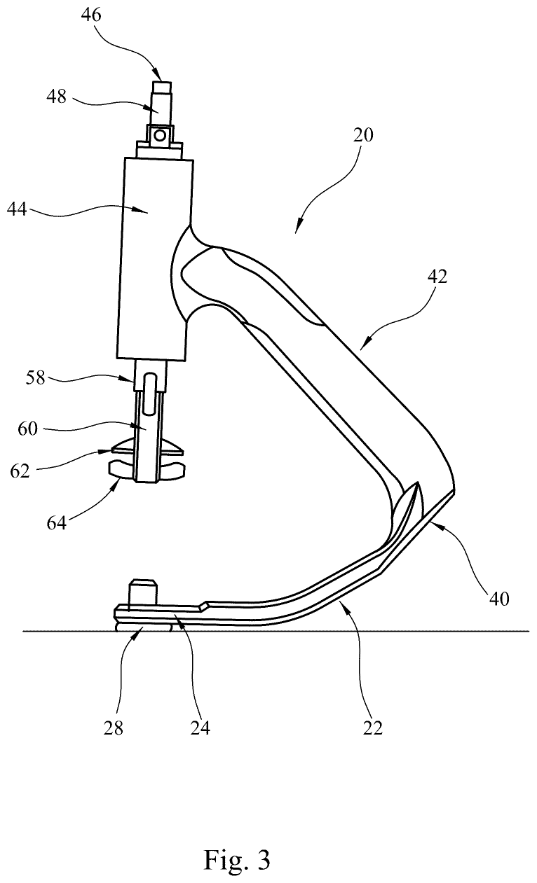

Returning to FIG. 3, the remining features of the instrument 20 are described. A handle element 42 links the foot section 22 to the shaft mounting section 44. The handle element 42 is profiled to be an ergonomic fit with a left and/or right hand of the user. The handle element 42 is generally perpendicular to the third planar base section 40. The configuration of the handle element 42 allows the user to firmly push the instrument 20 into engagement with the clamping ring 28 or implant stem [not shown]. The configuration of the handle element 42 also allows the user to flex their wrist and provide fine adjustment to the orientation.

The shaft mounting section 44 is generally cylindrical. The shaft mounting section 44 extends distally relative to the handle element 42 towards the forked section 24. The shaft mounting section 44 also extends proximally relative to the handle element 42.

The drive mechanism, for instance powered screw drive 50, connects to the distal end 46 of a first driven shaft 48. The driven shaft 48 extends into the shaft mounting section 44 and connects to a second driven shaft 58. Within the shaft mounting section 44, rotational drive of the first driven shaft 48 is converted to axial movement of the second driven shaft 58. Driving the first driven shaft 48 in one direction advances the second driven shaft 58 towards the forked section 24. Reversing the drive direction retracts the second driven shaft 58 away from the forked section 24. The distal end 60 of the second driven shaft 58 is provided with a liner receiving cup 62 which has an internal distal profile adapted to match a part of the proximal external profile of the liner [not shown in this figure]. Retaining element 64 serves to retain the liner in the liner receiving cup 62.

The use location 100 for the instrument 20 and drive mechanism 50 is the position of the user's hand 102 when on the instrument's user interface, typically the handle 104. As described below, with reference to FIG. 4, a wide variety of factors influence where this use location 100 is spatially within the environment 106.

The three different states A, B, C, for the instrument 20 described above allow the instrument 20 to be successfully and comfortably operated, for its intended purpose, in a variety of situations. The variety of situations can be thought of as covering the different situations which arise with different values for multiple factors which have an influence on the use environment. Such factors are at least in part identified in FIG. 4, albeit with reference to a single state instrument 20 for clarity. The multiple factors include those which influence the use location 100 for the instrument 20 and alternate possible use locations with different states [not shown].

A key set of factors are all those which have an influence on the height of the use location 100 relative to the floor 108 of the environment 106 where the instrument 20 is being used. The total inferior to superior height can include a number of different factors. These include:

The floor 108 to surgical table top 32 factor, here defined as the floor to table length 110, which contributes a significant portion of this height variation. A wide range of values between different table top 32 may be encountered, for instance heights of 900 mm to 1200 mm are common.

The head length 112 is the next factor encountered. This will vary with the head design employed and hence the physical dimensions of that head. Typical values for this factor could centre around or have a maximum of 25 mm.

The liner length 114 is the next factor moving superiorly. This will vary with the type of liner selected for use and the different dimensions that come with that and the different separation which is needed to introduce the liner to the head.

The stroke length 116 is the next factor and relates to the separation needed to align the liner with the head and then reduce towards one another. This will vary with head and liner designs but may be around 80 mm.

Finally, the drive mechanism configuration, identified herein as the gun width 118, will further influence where the use location 100 is.

If the instrument 20 was in State A, then the use location 100 will move inward over the table 34 and superiorly, arrow A. If the instrument was in State C, then the use location 100 will move outward from the table 34 and inferiorly, arrow C.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the apparatus, system, and method described herein. It will be noted that alternative embodiments of the apparatus, system, and method of the present disclosure may not include all of the features described yet still benefit from at least some of the advantages of such features. Those of ordinary skill in the art may readily devise their own implementations of the apparatus, system, and method that incorporate one or more of the features of the present invention and fall within the spirit and scope of the present disclosure.

1. A surgical instrument, the surgical instrument comprising:
a handle element;
a driven element mounting section connected to the handle element;
one or more driven elements, the one or more driven elements including a driven element providing a proximal driver attachment location, the one or more driven elements including a driven element providing a proximal implant component contactor; and
an instrument support section, the instrument support section including a first abutment surface and at least a second abutment surface;
wherein the instrument has a driven axis, the driven axis corresponding to a driven element axis extending distally to proximally through one or more of the driven elements;
wherein a first intersection defined between the driven axis and the first abutment surface forms a first angle and a second intersection defined between the driven axis and the second abutment surface forms a second angle, the second angle being different to the first angle.

2. An instrument according to claim 1, wherein the instrument has a first state in which the first abutment surface is adapted to contact an instrument support location, such as an instrument support table surface, and the instruments has a second state in which the second abutment surface is adapted to contact an instrument support location.

3. An instrument according to claim 2, wherein the instrument has a first state in which the driven axis has a first angular orientation relative to the vertical and the instrument has a second state in which the driven axis has a second, different, angular orientation relative to the vertical.

4. An instrument according to claim 3, wherein the instrument has a third state in which a third abutment surface is adapted to contact an instrument support location.

5. An instrument according to claim 3, wherein the instrument has a third state in which the driven axis has a third, different, angular orientation relative to the vertical.

6. An instrument according to claim 1, having a first state in which the driven axis is vertical +/−5° and/or having a second state in which the driven axis is at 30° to the vertical +/−5° and/or having a third state in which the driven axis is at 45° to the vertical +/−5° and/or having a further state in which the driven axis is off set from the vertical by more than 55° to the vertical +/−5°.

7. An instrument according to claim 1, wherein the instrument support section includes a first abutment surface and a second abutment surface and a third abutment surface.

8. An instrument according to claim 7, wherein within the instrument support section, the first abutment surface is more distal to the handle element than the second abutment surface and/or third abutment surface and/or wherein within the instrument support section, the second abutment surface is more distal to the handle element than the third abutment surface and/or more proximal to the handle element than the first abutment surface.

9. An instrument according to claim 7, wherein at least one of the first abutment surface, the second abutment surface, and the third abutment surface include a substantially planar surface section.

10. An instrument according to claim 7, wherein a first abutment surface is inclined relative to a second abutment surface by an angle of 30°+/−10° and/or a first abutment surface is inclined relative to a third abutment surface by an angle of 45°+/−10 °.

11. An instrument according to claim 1, wherein the driven element mounting section is integrally provided with the handle element and integrally provided with the instrument support section.

12. An instrument according to claim 1, wherein the distal implant component contactor is adapted to receive a prosthesis component, such as a liner and/or wherein the instrument support section is connected to a second implant component contactor adapted to receive a second prosthesis component, such as a head.

13. A kit, the kit comprising:
a surgical instrument;
a driver device;
wherein the surgical instrument comprises:
a handle element;
a driven element mounting section connected to the handle element;
one or more driven elements, the one or more driven elements including a driven element providing a proximal driver attachment location, the one or more driven elements including a driven element providing a proximal implant component contactor; and
an instrument support section, the instrument support section including a first abutment surface and at least a second abutment surface;
wherein the instrument has a driven axis, the driven axis corresponding to a driven element axis extending distally to proximally through one or more of the driven elements;
wherein a first intersection defined between the driven axis and the first abutment surface forms a first angle and a second intersection defined between the driven axis and the second abutment surface forms a second angle, the second angle being different to the first angle.

14. A kit according to claim 13, wherein the driver device is a rotary driver device or a ratchet.

15. A method of assembly of a multi-component surgical implant, the method comprising:
a) providing at least two components of the multi-component surgical implant;
b) providing a surgical instrument comprising:
i. a handle element;
ii. a driven element mounting section connected to the handle element;
iii. one or more driven elements, the one or more driven elements including a driven element providing a proximal driver attachment location, the one or more driven elements including a driven element providing a proximal implant component contactor; and
iv. an instrument support section, the instrument support section including a first abutment surface and at least a second abutment surface;
v. wherein the instrument has a driven axis, the driven axis corresponding to a driven element axis extending distally to proximally through one or more of the driven elements;
vi. wherein a first intersection defined between the driven axis and the first abutment surface forms a first angle and a second intersection defined between the driven axis and the second abutment surface forms a second angle, the second angle being different to the first angle;
c) providing a first component of the surgical implant on the instrument support section;
d) providing a second components of the surgical implant on the proximal implant component contactor;
e) abutting a chosen one of the abutment surfaces with a non-instrument surface;
f) driving a driven element to reduce the separation of the second component relative to the first component and push the second component and the first component into an assembled form.

16. The method of claim 15, wherein the instrument support section supports the instrument in cooperation with the non-instrument surface, such as a table surface.

17. The method of claim 15, wherein the method includes moving the instrument to change the abutment surface abutting with the non-instrument surface.

18. The method of any of claims 15, wherein the method includes connecting a driver device to the proximal driver attachment location and/or activating a driver device to drive a driven element.

19. The method of any of claims 15, wherein a driver device is provided and includes a driver handle section that defines a user location in the environment of the instrument and the method includes changing the abutment location used to change the user location in the environment of the instrument.

20. The method of claim 19, wherein the method includes changing the abutment location used to change the inclination of the driven axis.

21. A method of surgery using a multi-component surgical implant, the method comprising:
a) providing at least two components of the multi-component surgical implant;
b) providing a surgical instrument comprising:
i. a handle element;
ii. a driven element mounting section connected to the handle element;
iii. one or more driven elements, the one or more driven elements including a driven element providing a proximal driver attachment location, the one or more driven elements including a driven element providing a proximal implant component contactor; and
iv. an instrument support section, the instrument support section including a first abutment surface and at least a second abutment surface;
v. wherein the instrument has a driven axis, the driven axis corresponding to a driven element axis extending distally to proximally through one or more of the driven elements;
vi. wherein a first intersection defined between the driven axis and the first abutment surface forms a first angle and a second intersection defined between the driven axis and the second abutment surface forms a second angle, the second angle being different to the first angle;
c) providing a first component of the surgical implant on the instrument support section;
d) providing a second components of the surgical implant on the proximal implant component contactor;
e) abutting a chosen one of the abutment surfaces with a non-instrument surface;
f) driving a driven element to reduce the separation of the second component relative to the first component and push the second component and the first component into an assembled form.
